(19) United States
(12) Patent Application Publication
(10) Pub. No.: US 2013/0190876 A1
(43) Pub. Date: Jul. 25, 2013

(54) EXPANDABLE INTERBODY IMPLANT AND METHODS OF USE

(71) Applicants: Thomas E. Drochner, Memphis, TN (US); Keith E. Miller, Germantown, TN (US)

(72) Inventors: Thomas E. Drochner, Memphis, TN (US); Keith E. Miller, Germantown, TN (US)

(73) Assignee: WARSAW ORTHOPEDIC, INC., Warsaw, IN (US)

(21) Appl. No.: 13/354,002

(22) Filed: Jan. 19, 2012

(51) Int. Cl. A61F 2/44

(52) U.S. Cl. 623/17.16

(57) ABSTRACT

An intervertebral implant comprises a first component and a second component. A first arm extends between a first end and a second end. A second arm extends between a first end and a second end. The first ends of the arms are engageable with the first component. An actuator is disposed within the second component and includes a first member and a second member. The members are configured for axial translation relative to the actuator. The first member translates in a first axial direction and is engageable with the first arm and the second member translates in a second axial direction and is engageable with the second arm such that the arms move the first component and the second component between a first, collapsed configuration and a second, expanded configuration. Methods of use are disclosed.

### TECHNICAL FIELD

The present disclosure generally relates to medical devices, systems and methods for the treatment of musculoskeletal disorders, and more particularly, to an interbody implant system and method for treating a vertebral column.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. These treatments may employ interbody implants. This disclosure describes an improvement over these prior art technologies.

### SUMMARY

Accordingly, an expandable interbody implant system and method are disclosed. In one embodiment, an intervertebral implant is provided. The intervertebral implant comprises a first component and a second component. A first arm extends between a first end and a second end. A second arm extends between a first end and a second end. The first ends of the arms are engageable with the first component. An actuator is disposed with the second component and includes a first member and a second member. The members are configured for axial translation relative to the actuator. The first member translates in a first axial direction and is engageable with the first arm and the second member translates in a second axial direction and is engageable with the second arm such that the arms move the first component and the second component between a first, collapsed configuration and a second, expanded configuration.

In one embodiment, the intervertebral implant comprises a posterior component extending between a first end and a second end. The posterior component includes an outer surface and an inner surface. The posterior component defines a first slot and a second slot. An anterior component extends between a first end and a second end. The anterior component includes an outer surface and an inner surface. A linkage includes a first arm and a second arm. The first arm includes a first end configured for axial movement within the first slot and a second end connected with the anterior component. The second arm includes a first end configured for axial movement within the second slot and a second end connected with the anterior component. An actuator includes a longitudinal screw disposed with the anterior component. A first threaded collar is disposed with the screw and a second threaded collar is disposed with the screw. The screw is rotatable to translate the first collar in a first axial direction to engage the first arm such that the first arm rotates relative to the anterior component and to translate the second collar in a second axial direction to engage the second arm such that the second arm rotates relative to the anterior component such that the first component and the second component are movable in a first transverse orientation relative to the longitudinal axis between a first collapsed configuration and a second expanded configuration, and the anterior component is movable in a second transverse orientation between a first collapsed height and a second expanded height.

In one embodiment, a method for treating a spine is provided. The method comprises the steps of: providing an intervertebral implant comprising: a first component, a second component, a first arm extending between a first end and a second end, a second arm extending between a first end and a second end, the first ends of the arms being engageable with the first component, and an actuator disposed with the second component and including a first member and a second member, the members being configured for axial translation relative to the actuator; introducing the intervertebral implant in a first, collapsed configuration along a substantially lateral approach of a body within an intervertebral space; and engaging the actuator such that the first member translates in a first axial direction and is engageable with the first arm and the second member translates in a second axial direction and is engageable with the second arm such that the arms move the first component and the second component between the first, collapsed configuration and a second, expanded configuration.

### BRIEF DESCRIPTION OF THE DRAWINGS

The present disclosure will become more readily apparent from the specific description accompanied by the following drawings, in which:

FIG. 1 is a perspective view of one particular embodiment of an implant of a system in accordance with the principles of the present disclosure;

FIG. 2 is a perspective view of the implant shown in FIG. 1;

FIG. 3 is a top cross section view of the implant shown in FIG. 1;

FIG. 4 is a top cross section view of the implant shown in FIG. 1;

FIG. 5 is a top cross section view of the implant shown in FIG. 1;

FIG. 6 is an end view of the implant shown in FIG. 1;

FIG. 7 is an end view of the implant shown in FIG. 1;

FIG. 8 is a plan view of components of a system in accordance with the principles of the present disclosure disposed with vertebrae;

FIG. 9 is a side view of components of the system and vertebrae shown in FIG. 8;

FIG. 10 is a plan view of components of the system and vertebrae shown in FIG. 8;

FIG. 11 is a side view of components of the system and vertebrae shown in FIG. 8;

FIG. 12 is a perspective view of one embodiment of the implant shown in FIG. 1;

FIG. 13 is a perspective view of the implant shown in FIG. 12;

FIG. 14 is a top cross section view of the implant shown in FIG. 12;

FIG. 15 is a top cross section view of the implant shown in FIG. 12;

FIG. 16 is a top cross section view of the implant shown in FIG. 12;

FIG. 17 is a front cross section view of the implant shown in FIG. 12;

FIG. 18 is a front cross section view of the implant shown in FIG. 12;

FIG. 19 is a front cross section view of the implant shown in FIG. 12;

FIG. 20 is an end view of the implant shown in FIG. 12; and

FIG. 21 is an end view of the implant shown in FIG. 12.

### DETAILED DESCRIPTION

The exemplary embodiments of an expandable interbody implant system and related methods of use disclosed herein are discussed in terms of medical devices for the treatment of musculoskeletal disorders and more particularly, in terms of an expandable interbody implant system and related methods for treating a vertebral column. It is envisioned that the implant system may provide, for example, fusion, decompression, restoration of sagittal balance and resistance of subsidence into tissue, such as, for example, surfaces of vertebral endplates. It is further envisioned that the system includes an interbody implant that expands after insertion into an intervertebral disc space and has several features, such as, for example, to facilitate insertion into the intervertebral disc space, decompression of nerve roots, expansion to restore sagittal balance such that more expansion is provided on an anterior side relative to a posterior side. In one embodiment, the expandable interbody implant system expands vertically to increase height and add lordosis, and expand laterally to increase width. This configuration maintains a large graft footprint.

In one exemplary application, a surgical practitioner employs a direct lateral interbody fusion (DLIF) procedure and operates through a window in a psoas muscle of a patient. It is envisioned that during the procedure, the surgical practitioner attempts to avoid the lumbar plexus, which passes through the psoas muscle, to avoid complications during surgery. It is further envisioned that the surgical practitioner targets an anterior portion of the psoas muscle to avoid the lumbar plexus. The expandable interbody implant system and related methods of use disclosed are configured to avoid these complications and allow the surgical practitioner to create a smaller surgical target window in an anterior position in the psoas muscle.

For example, during a surgical procedure, the surgical practitioner accesses an intervertebral disc space through the psoas muscle. An expandable interbody implant of the system is expanded from an anterior to a posterior direction in an anterior-posterior expansion. The expandable interbody implant provides a large footprint that improves stability and decreases the risk of subsidence into tissue. It is contemplated that the expandable interbody implant provides a low profile width implant that can be delivered through an opening in the psoas muscle, the opening being of reduced dimension, for example, one half of the width of the expanded configuration of the implant width. In the expanded configuration, the implant provides a surface for post-packing the implant with bone graft.

The expandable interbody implant system and related methods of use disclosed facilitate a DLIF procedure by creating a surgical target window in the psoas muscle that avoids nerves adjacent a surgical site. It is contemplated that the present implant system and method provide height and/or lordotic expansion that avoids damage to vertebral endplates during insertion. It is further contemplated that the present implant system and method provide height and/or lordotic expansion that facilitate decompression of nerve roots. It is envisioned that the present implant system and method provide an anterior-posterior (AP) expansion that provides a footprint to increase stability and reduce risk of subsidence into tissue. It is further envisioned that the expandable interbody implant system can be employed with a posterolateral interbody fusion (PLIF) and/or a transforaminal interbody fusion (TLIF), for example, which may require medial-lateral expansion of an implant.

In one embodiment, the expandable interbody implant system employs opposite handed threading to move a plurality of collars along a threaded rod actuator. The threaded mating configuration of the collars and the rod actuator is such that the collars are spaced apart and caused to converge in translation adjacent a mid portion and/or intermediate location of the implant upon rotation of the rod actuator. The implant includes a linkage attached to the collars for expanding the implant. The linkage includes arms, each having a geared section that interact during implant expansion. This gearing section prevents a posterior component of the implant from rotating and cause the components of the implant to expand in a linear AP and/or posterior-anterior (PA) expansion. In one embodiment, the implant has a width of 12 millimeters (mm) in the collapsed configuration and expands to a width of 22.5 mm in the expanded configuration. In one embodiment, the implant has a width in the range of 8-18 mm in the collapsed configuration.

In one embodiment, the expandable interbody implant includes a posterior component having a height shorter than an anterior component to induce lordosis of adjacent vertebrae. In one embodiment, the posterior component has an equal height as the anterior component.

In one embodiment, the expandable interbody implant employs moving wedges to facilitate expansion between the collapsed and expanded configurations. The wedges engage inner surfaces of the posterior and anterior components to increase height in the anterior component while engaging a linkage to drive the posterior component apart from the anterior component to create a larger footprint. In one embodiment, the wedge pushes the posterior component apart from the anterior component via arms of the linkage, which slide in slots formed in the posterior component. The implant employs opposite handed threading such that the wedges and an actuator are threaded. The wedges are disposed at a central position along the actuator and space apart to expand the components. In one embodiment, the implant has a width of 12 mm in the collapsed configuration and expands to a width of 21 mm in the expanded configuration. In one embodiment, the wedges drive height expansion in the anterior component. For example, the anterior component expands in height from 10 mm to 12.5 mm.

It is envisioned that the expandable interbody implant and methods of use disclosed herein can be employed to obtain fusion of vertebrae through a minimally invasive or percutaneous technique. In one embodiment, the disclosed expandable interbody implant and methods of use can provide improved spinal treatment with a device that is made to expand vertically to create lordosis in vertebrae. It is contemplated that the expandable interbody implant and methods of use disclosed herein provide a cavity of relatively large volume for post-packing of at least one agent, for example, bone graft.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed expandable interbody implant may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The expandable interbody implant of the present disclosure may also be alternatively employed with procedures for treating the lumbar, cervical, thoracic and pelvic regions of a spinal column. The expandable interbody implant and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, outer, inner, terminal (denoting position or location), left and right, posterior, anterior, and the like, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (for example, preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, for example, arresting its development, or relieving the disease, for example, causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

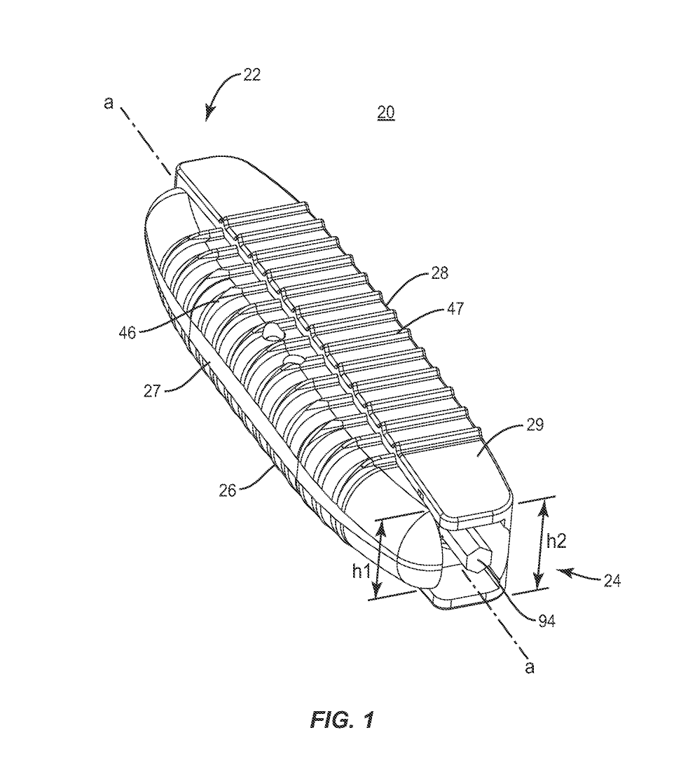

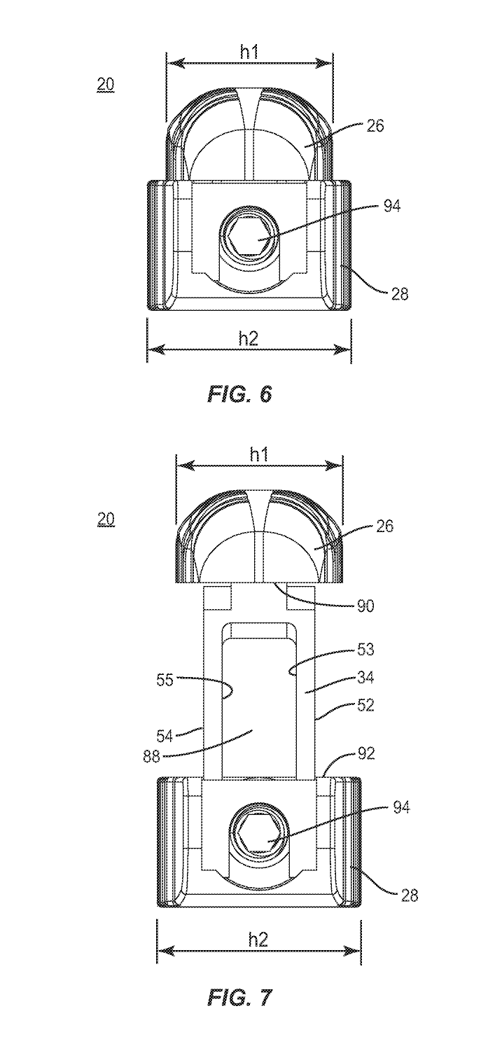

The following discussion includes a description of an expandable interbody implant and related methods of employing the expandable interbody implant in accordance with the principles of the present disclosure. Alternate embodiments are also disclosed. Reference will now be made in detail to the exemplary embodiments of the present disclosure, which are illustrated in the accompanying figures. Turning now to FIGS. 1-7, there is illustrated components of an interbody implant system including an intervertebral implant 20 in accordance with the principles of the present disclosure.

The components of the system can be fabricated from biologically acceptable materials suitable for medical applications, including metals, synthetic polymers, ceramics and bone material and/or their composites, depending on the particular application and/or preference of a medical practitioner. For example, the components of the system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (for example, Nitinol, super elasto-plastic metals, such as GUM METAL® manufactured by Toyota Material Incorporated of Japan), ceramics and composites thereof such as calcium phosphate (for example, SKELITE™ manufactured by Biologix Inc.), thermoplastics such as polyaryl ether ketone (PAEK) including polyether ether ketone (PEEK), polyether ketone ketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation fitctors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polylactide, polyglycolide, polytyrosine carbonate, polycaprolactone and their combinations. Various components of the system may be fabricated from material composites, including the above materials, to achieve various desired characteristics such as strength, rigidity, elasticity, flexibility, compliance, biomechanical performance, durability and radiolucency or imaging preference. The components of the system, individually or collectively, may also be fabricated from a heterogeneous material such as a combination of two or more of the above-described materials.

The system including intervertebral implant 20 can be employed as a stabilization device in fusion and fixation procedures, for example, for patients suffering from a spinal disorder to provide height restoration between vertebral bodies, decompression, restoration of sagittal balance and/or resistance of subsidence into vertebral endplates. The components of the interbody implant system may be monolithically formed, integrally connected or include fastening elements and/or instruments, for example, as described herein.

Intervertebral implant 20 defines a longitudinal axis a and extends between a first end 22 and a second end 24. Intervertebral implant 20 includes a first component, such as, for example, a posterior component 26 and a second component, such as, for example, an anterior component 28 connected to posterior component 26. Posterior component 26 is movably mounted to anterior component 28. Posterior component 26 has a first height h1 and anterior component 28 has a second height h2 greater then height h1. It is contemplated that height h1 can be greater than height h2 or height h1 can equal height h2. It is further contemplated that implant 20 has a width of approximately 12 millimeters (mm) in the collapsed configuration and expands to a width of 22.5 mm in the expanded configuration. It is contemplated that implant 20 has a width in the range of 8-18 mm in the collapsed configuration. It is further contemplated that implant 20 has a width in the range of 18-30 mm in the expanded configuration.

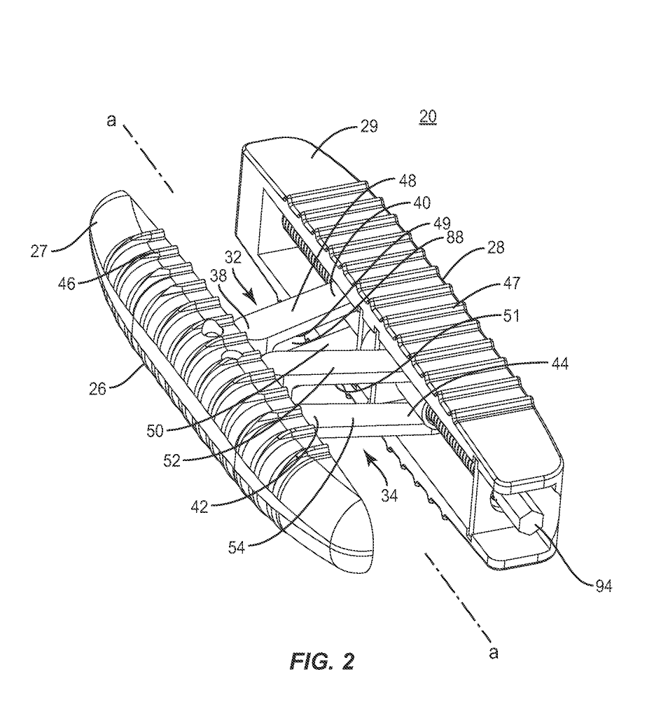

Posterior component 26 has a tapered configuration from intermediate the component to ends 22, 24. Posterior component 26 includes an inner surface 90 that defines a cavity Posterior component 26 includes an outer surface 27 configured to engage an endplate of a vertebra and includes a plurality of raised elements, such as, for example, teeth 46 configured to enhance fixation and/or gripping with vertebral tissue. Teeth 46 are disposed transverse to longitudinal axis a. Outer surface 27 is configured for engagement with the upper and lower vertebrae. As shown in FIGS. 1 and 2, teeth 46 are provided along the outer surface 27.

However, other engagement surfaces can be employed such as, for example, rough, arcuate, undulating, mesh, porous, semi-porous, dimpled and/or textured according to the requirements of a particular application. It is also contemplated that the posterior component 26 may be coated with biocompatible materials such as an osteoconductive material such as (HA)-TCP and/or osteoinductive agent such as a bone morphogenic protein (BMP) for enhanced bony fixation. It is envisioned that the biocompatible material and/or an agent employed with an implant of the spinal rod system may include one or more therapeutic agent(s) disposed in one or more layers or homogenously throughout it.

Anterior component 28 includes an inner surface 92 that defines a cavity 93. Anterior component 28 has an outer surface 29 configured to engage an endplate of a vertebra and includes a plurality of raised elements, such as, for example teeth 47 configured to enhance fixation and/or gripping with vertebral tissue. Teeth 47 are disposed transverse to longitudinal axis a. Outer surface 29 is configured for engagement with the upper and lower vertebrae. As shown in FIGS. 1 and 2, teeth 47 are provided along the outer surface 29. However, other engagement surfaces can be employed such as, for example, rough, arcuate, undulating, mesh, porous, semi-porous, dimpled and/or textured according to the requirements of a particular application. It is also contemplated that the anterior component 28 may be coated with biocompatible materials such as an osteoconductive material such as (HA)-TCP and/or osteoinductive agent such as a bone morphogenic protein (BMP) for enhanced bony fixation. It is envisioned that the biocompatible material and/or an agent employed with implant 20 may include one or more therapeutic agent(s) disposed in one or more layers or homogenously throughout it

Posterior and anterior components 26, 28 are relatively movable to expand and collapse between a first configuration and a second configuration, as will be described below. Posterior and anterior components 26, 28 are movable by a linkage, which includes a first arm 32 and a second arm 34.

First arm 32 extends between a first end 38 and a second end 40. Second arm 34 extends between a first end 42 and a second end 44. It is contemplated that components 26, 28 may be monolithically formed and/or be connected via a living hinge. It is further contemplated that anterior component 28 may be alternatively connected to posterior component 26 by integral connection, and/or fastening elements such as clips and/or screws.

First arm 32 comprises a first strut 48 and a second strut 50 disposed in parallel orientation. It is contemplated that struts 48, 50 can have alternate orientations, relative to longitudinal axis a, for example, perpendicular, converging, diverging and/or other angular orientations such as acute or obtuse, co-axial and/or may be offset or staggered and may extend in alternate configurations, such as, for example, radius of curvature, offset and/or staggered. It is further envisioned that struts 48, 50 may have various cross section configurations, such as, for example, oval, oblong, triangular, rectangular, square, polygonal, irregular, uniform, non-uniform, variable, hollow and/or tapered. Struts 48, 50 include inner surfaces 49 and 51, respectively. Inner surfaces 49, 51 define a bone graft receptacle 88. As shown in FIG. 7, bone graft receptacle 88 is rectangular in shape. It is contemplated that bone graft receptacle can be any shape such as, for example, oval, oblong, triangular, rectangular, square, polygonal, irregular, uniform, non-uniform, variable, hollow and/or tapered.

Second arm 34 includes a first strut 52 and a second strut 54 disposed in parallel orientation. It is contemplated that struts 52, 54 can have alternate orientations, relative to longitudinal axis a, for example, perpendicular, converging, diverging and/or other angular orientations such as acute or obtuse, co-axial and/or may be offset or staggered and may extend in alternate configurations such as, for example, radius of curvature, offset and/or staggered. It is further envisioned that struts 48, 50 may have various cross section configurations, such as, for example, oval, oblong, triangular, rectangular, square, polygonal, irregular, uniform, non-uniform, variable, hollow and/or tapered. Struts 52, 54 include inner surfaces 53 and 55, respectively. Inner surfaces 53, 55 define a bone graft receptacle 90. As shown in FIG. 7, bone graft receptacle 90 is rectangular in shape. It is contemplated that bone graft receptacle can be any shape such as, for example, oval, oblong, triangular, rectangular, square, polygonal, irregular, uniform, non-uniform, variable, hollow and/or tapered.

First arm 32 is rotatably connected to posterior component 26 via pivot pin 66 for pivotal movement relative thereto. It is contemplated that arm 32 is rotatable relative to component 26 through an angle of 0 to 90 degrees. Second arm 34 is rotatably connected to posterior component 26 via pivot pin 66 for pivotal movement relative thereto. It is contemplated that arm 34 is rotatable relative to component 26 through an angle of 0 to 90 degrees. Pivot pins 66 allow first and second arms 32, 34 to rotate about posterior component 26.

First end 38 of first arm 32 includes a geared surface 56 engageable with a geared surface 58 disposed at the first end 40 of second arm 34. Geared surfaces 56, 58 interact with each other as implant 20 is expanding. Geared surfaces 56, 58 prevent posterior component 26 from rotating and allows posterior component 26 to move linearly during expansion. First arm 32 is rotatable in a clockwise direction relative to the posterior component 26 and the second arm 34 is rotatable in a counterclockwise direction relative to the posterior component 26 to move the posterior component 26 and the anterior component 28 between the first, collapsed configuration and the second, expanded configuration. It is contemplated that first end 38 and first end 42 may have alternative surface configurations, such as, for example, rough, arcuate, undulating, dimpled and/or textured according to the requirements of a particular application.

First and second arms 32, 34 are moveable by actuation of an actuator, such as, for example, a screw 94 disposed in the anterior component 28. Screw 94 includes an outer threaded surface Screw 94 extends between first end 22 and second end 24 and is mounted in cavity 82 of anterior component 28. Screw 94 includes a first member, such as, for example, a collar 60 and a second member, such as, for example, a collar 62.

Collar 60 has a tubular configuration with a threaded cylindrical inner surface 61. It is contemplated that collar 60 can have alternate configurations, such as, oval, oblong, triangular, rectangular, square, polygonal, irregular, uniform, non-uniform, variable, hollow and/or tapered. It is contemplated that inner surface 61 can be smooth, even, arcuate, undulating and/or textured according to the requirements of a particular application. Collar 60 is engageable with second shaft portion 94b of screw 94 and is connected with second end 40 of first arm 32. It is envisioned that connection with second end 40 includes a pin, hinge or living hinge.

Collar 62 has a tubular configuration with a threaded cylindrical inner surface 63. It is contemplated that collar 62 can have alternate configurations, such as, oval, oblong, triangular, rectangular, square, polygonal, irregular, uniform, non-uniform, variable, hollow and/or tapered. It is contemplated that inner surface 63 can be smooth, even, arcuate, undulating and/or textured according to the requirements of a particular application. Collar 62 is engageable with first shaft portion 94a of screw 94 and is connected with second end 44 of first arm 34. It is envisioned that connection with second end 44 includes a pin, hinge or living hinge.

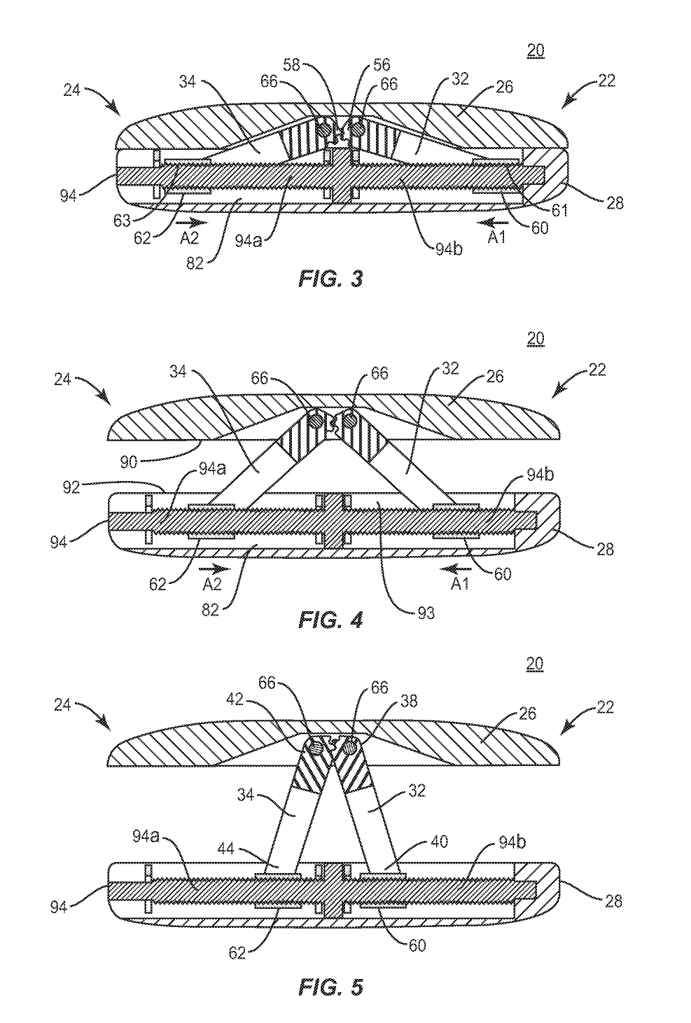

In one embodiment, collar 60 has a first direction threading and collar 62 has an opposite direction threading to move collars 60, 62 along screw 94. In one embodiment, first shaft portion 94a has a first direction threading and second shaft portion 94b has an opposite direction threading to move collars 60, 62 along screw 94. The threaded mating configuration of collars 60, 62 and screw 94 is such that collars 60, 62 are spaced apart and caused to converge in translation adjacent a mid portion and/or intermediate location of anterior component 28. Collar 60 is configured for axial movement in a first axial direction, as shown by arrow A1 in FIGS. 3 and 4, and collar 62 is configured for axial movement in a second axial direction, as shown by arrow A2. Collars 60, 62 translate along screw 94 in a converging relation such that arms 32, 34 rotate, as described above, to move posterior component 26 and anterior component 28 between a first, collapsed configuration and a second, expanded configuration.

In operation, as shown in FIGS. 3-7, intervertebral implant 20 is engaged for disposal between a first configuration and a second configuration such that intervertebral implant 20 expands in an intervertebral disc space. Intervertebral implant 20 is engaged with an instrument (not shown) to facilitate actuation of the component parts of intervertebral implant 20, according to the requirements of a particular surgical application.

In a first configuration, such as for example, a collapsed configuration, as shown in FIG. 3, components 26, 28 are disposed in a low profile orientation such that collar 60 is disposed adjacent first end 22 and collar 62 is disposed adjacent second end 24. Posterior and anterior components 26, 28 are disposed in an engaging configuration such that the respective end surfaces of components 26, 28 are in flush contact.

Upon desired positioning of intervertebral implant 20, according to the requirements of a particular surgical application, screw 94 is manipulated to move collars 60, 62 axially. The instrument engages screw 94 for rotation in a clockwise direction. Screw 94 rotates such that outer surface 45 threadably engages inner surface 61 of collar 60 and inner surface 63 of collar 62. As screw 94 rotates, the threaded engagement with inner surfaces 61, 63 drive collars 60, 62 in opposing axial directions, as shown by arrows A1, A2. This axial movement of collars 60, 62 moves arms 32, 34 such that arms 32, 34 rotate about pivot pins 66 and relative to component 26. The linkage including arms 32, 34 cause component 26 to translate in a posterior direction relative to component 28 transverse to axis a to space apart components 26, 28. Intervertebral implant 20 is expanded from an anterior to a posterior direction in an anterior-posterior expansion. This configuration expands components 26, 28 between the first collapsed configuration, as shown in FIG. 3, and the second, expanded configuration, as shown in FIG. 5. Components 26, 28 expand laterally to increase width and, which may provide a large graft footprint. Posterior component 26 has height h1 and anterior component 28 has height h2, which is greater than height h1. This configuration can be employed to induce lordosis.

In use, as shown in FIGS. 8-11, the interbody implant system including intervertebral implant 20, similar to that described above with regard to FIGS. 1-7, is employed with a surgical procedure, such as, for example, a fusion treatment of a spine of a patient including vertebrae V, intervertebral disc space I and body areas adjacent thereto. The interbody implant system may also be employed with other surgical procedures, such as, for example, discectomy, laminotomy, laminectomy, nerve root retraction, foramenotomy, facetectomy, decompression, and spinal, nucleus or disc replacement.

For example, intervertebral implant 20 can be employed with a surgical arthrodesis procedure, such as, for example, a DLIF procedure for treatment of an applicable condition or injury of an affected section of a spinal column and adjacent areas within a body, such as, for example, intervertebral disc space I between a first vertebra V1 and a second vertebra V2 of vertebrae V. It is contemplated that intervertebral implant 20 of the interbody implant system can be inserted with intervertebral disc space I to space apart articular joint surfaces, provide support and maximize stabilization of vertebrae V. It is further contemplated that intervertebral implant 20 provides height restoration between vertebral bodies, decompression, restoration of sagittal balance and/or resistance of subsidence into vertebral endplates.

In use, to treat the affected section of vertebrae V, a medical practitioner obtains access to a surgical site including vertebrae V in any appropriate manner, such as through incision and retraction of tissues. It is envisioned that the interbody implant system can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure is performed for treating the spine disorder. Intervertebral implant 20 is then employed to augment the surgical treatment. Intervertebral implant 20 can be delivered or implanted as a pre-assembled device or can be assembled in situ. Intervertebral implant 20 can be completely or partially revised, removed or replaced in situ. It is contemplated that one or all of the components of the interbody implant system can be delivered to the surgical site via manual manipulation and/or a free hand technique. It is further contemplated that intervertebral implant 20 may be inserted posteriorly, and then manipulated anteriorly and/or lateral and/or medial.

An incision is made in the body of a patient and a cutting instrument (not shown) creates a surgical pathway for implantation of intervertebral implant 20 within the patient body. The medical practitioner operates through the surgical pathway, which includes a window in a psoas muscle of the patient body. In one embodiment, the medical practitioner attempts to avoid the lumbar plexus, which passes through the psoas muscle, and nerves adjacent a surgical site, to avoid complications during surgery. In one embodiment, the medical practitioner targets an anterior portion of the psoas muscle to avoid the lumbar plexus. Intervertebral implant 20 is configured to avoid the above complications and allow the medical practitioner to create a smaller surgical target window in an anterior position in the psoas muscle.

A guide instrument (not shown) is employed to initially distract vertebra V1 from vertebra V2. A sleeve or cannula is used to access intervertebral disc space I and facilitate delivery and access for components of the interbody implant system. A preparation instrument (not shown) can be inserted within the sleeve or cannula and disposed within intervertebral disc space I. The preparation instrument(s) can be employed to remove some or all of the disc tissue including the disc nucleus and fluids, adjacent tissues and/or bone, corticate, scrape and/or remove tissue from the surfaces of endplates of opposing vertebrae V1, V2, as well as for aspiration and irrigation of the region, according to the requirements of a particular surgical application.

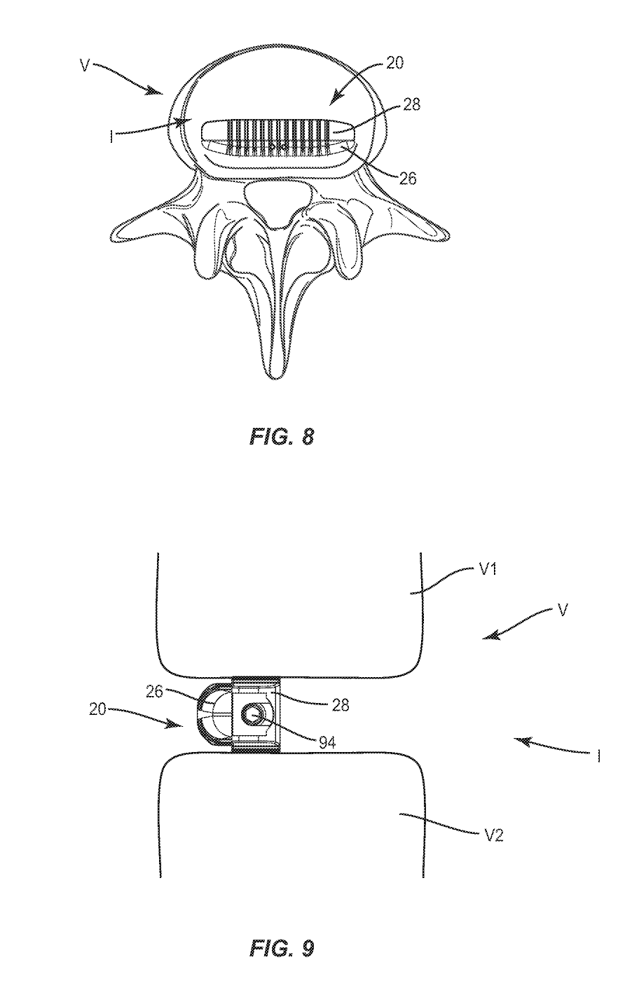

Intervertebral implant 20 is disposed in the first, collapsed configuration and delivered through the surgical pathway into intervertebral disc space I, as shown in FIGS. 8 and 9, with a delivery instrument (not shown) including a driver. The driver delivers intervertebral implant 20 into the prepared intervertebral disc space I, between vertebra V1 and vertebra V2, according to the requirements of a particular surgical application. It is contemplated that intervertebral implant 20 has a low profile width that can be delivered through an opening in the psoas muscle, the opening being of reduced dimension, for example, one half of the width of the expanded configuration of the width of intervertebral implant 20.

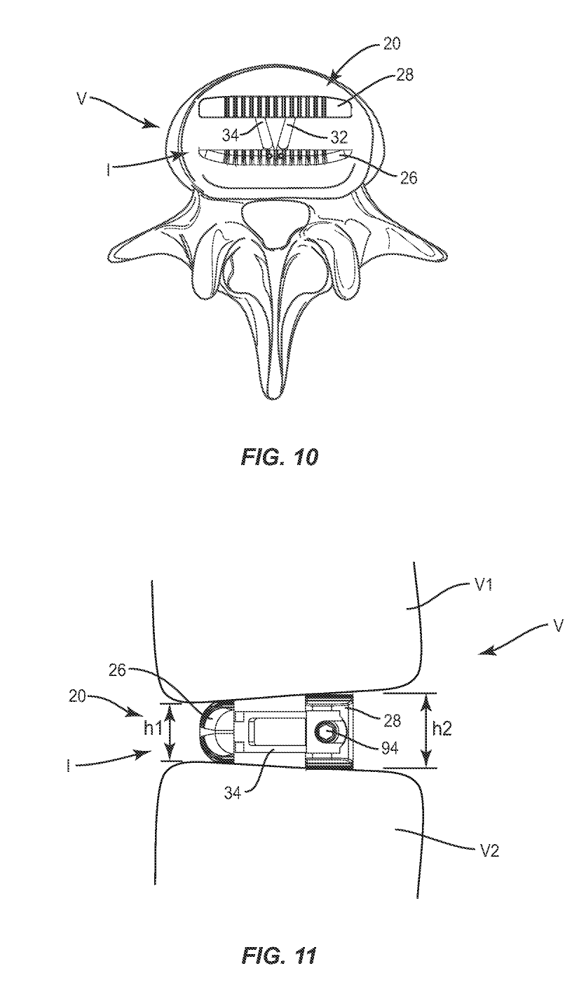

Upon desired positioning of intervertebral implant 20, screw 94 is manipulated to move collars 60, 62 axially. The delivery instrument engages screw 94 for rotation in a clockwise direction. As screw 94 rotates, collars 60, 62 are driven in opposing axial directions, as shown by arrows A1, A2. Arms 32, 34 rotate about pivot pins 66 and relative to component 26. The linkage including arms 32, 34 cause component 26 to translate in a posterior direction relative to component 28 to space apart components 26, 28. Intervertebral implant 20 is expanded from an anterior to a posterior direction in an anterior-posterior expansion. This configuration expands components 26, 28 between the first collapsed configuration and the second, expanded configuration, as shown in FIGS. 10 and 11. Components 26, 28 expand laterally to increase width. In the expanded configuration, intervertebral implant 20 provides a large footprint that improves stability and decreases the risk of subsidence into tissue. In the expanded configuration, the outer and/or inner surface of intervertebral implant 20 provides a surface for post-packing with bone graft.

Posterior component 26 has height h1 and anterior component 28 has height h2, which is greater than height h1, as shown in FIG. 11. This configuration provides height and/or lordotic expansion that avoids damage to vertebral endplates during insertion and facilitates decompression of nerve roots.

It is envisioned that the components of the interbody implant system, which may include one or a plurality of intervertebral implants 20, can be delivered to the surgical site via alternate approaches. In one embodiment, intervertebral implant 20 is delivered through the surgical pathway along a transforaminal lumbar interbody fusion approach, for example, which may require medial-lateral expansion into intervertebral disc space I. In one embodiment, intervertebral implant 20 is delivered through the surgical pathway along a posterior lumbar interbody fusion approach into intervertebral disc space I and disposed in the expanded configuration in a side by side orientation.

In one embodiment, intervertebral implant 20 can be collapsed from the expanded configuration to an alternate configurations between the expanded and collapsed configurations, as described above, to collapse intervertebral implant 20 as may be desired to reposition with or remove intervertebral implant 20 from intervertebral disc space I. In one embodiment, the interbody implant system includes a plurality of intervertebral implants 20, which can be variously sized and configured, and/or oriented in a side by side engagement, spaced apart and/or staggered.

In one embodiment, the interbody implant system includes an agent, which can include a bone growth promoting material, which may be disposed, packed or layered within, on or about the components and/or surfaces of the interbody implant system. The bone growth promoting material, such as, for example, bone graft can be a particulate material, which may include an osteoconductive material such as HA and/or an osteoinductive agent such as a bone morphogenic protein (BMP) to enhance bony fixation of intervertebral implant 20 with the adjacent vertebrae V.

It is contemplated that the agent and/or bone graft may include therapeutic polynucleotides or polypeptides. It is further contemplated that the agent and/or bone graft may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines.

Intervertebral implant 20 can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of the interbody implant system. Upon completion of the procedure, the surgical instruments and assemblies are removed and the incision is closed.

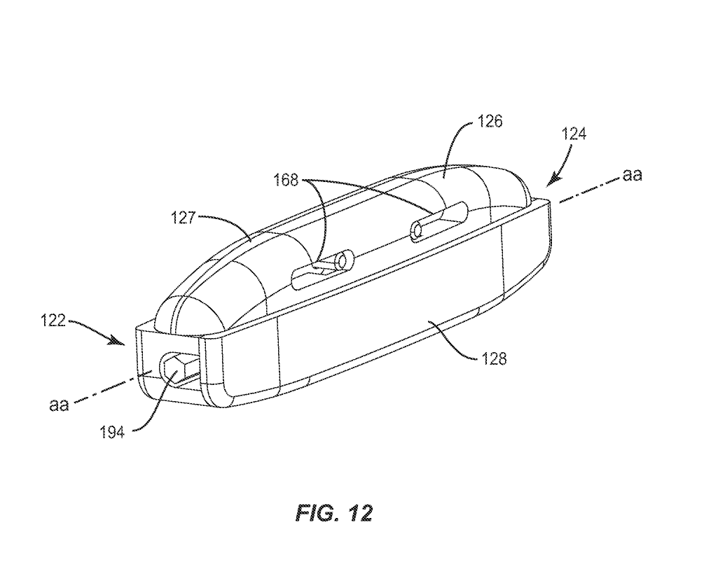

In one embodiment, as shown in FIGS. 12-21, the interbody implant system includes intervertebral implant 20, similar to the implant and methods of use described with regard to FIGS. 1-11, which defines a longitudinal axis as and extends between a first end 122 and a second end 124. Intervertebral implant 20 includes a first component, such as, for example, a posterior component 126 and a second component, such as, for example, an anterior component 128 connected to posterior component 126. Anterior component 128 is movably mounted to posterior component 126 for expansion in a first transverse orientation relative to axis aa, as will be described.

Posterior component 126 has a tapered configuration from intermediate the component 126 to ends 122, 124. Posterior component 126 includes an inner surface 190 that defines a cavity 191. Posterior component 126 includes an outer surface 127 configured to engage an endplate of a vertebra, similar to surface 27 described above. Posterior component 126 includes elongated slots 168 that communicate with cavity 191. Slots 168 are co-axial and disposed in parallel with axis aa. It is envisioned that slots 168 may be disposed in alternate relative orientations such as, for example, offset, staggered, transverse or perpendicular. Slots 168 are configured for movable disposal of arms 132 and 134, as discussed below.

Anterior component 128 includes an inner surface 193 that defines a cavity 192. Anterior component 128 has an outer surface 129 configured to engage an endplate of a vertebra, similar to surface 29 described above.

Anterior component 128 includes a first member 128a and a second member 128b. Member 128a is connected to member 128b via a slot and groove attachment such that members 128a, 128b are relatively movable and can translate in a spaced apart relation. Members 128a, 128b are movable in opposing directions along a second transverse orientation relative to axis as between a first collapsed height h3 and a second expanded height h4, as will be described below. Height h4 is greater than height h3. It is contemplated that components 126, 128 expand from a height h3 of 10 mm to a height h4 of 12.5 mm. It is contemplated that components 126, 128 have a height h3 in the range of 7-14 mm. It is further contemplated that components 126, 128 have a height h4 in the range of 9-18 mm.

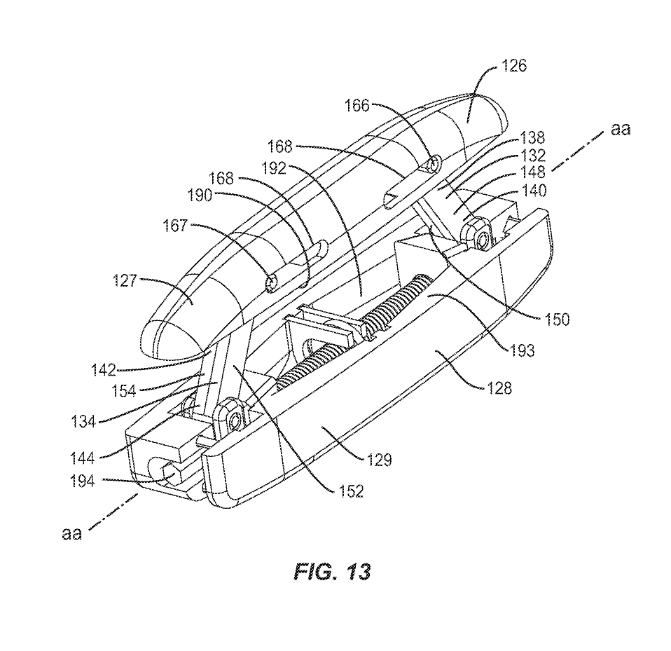

Posterior and anterior components 126, 128 are relatively movable to expand and collapse between a first configuration (FIG. 12) and a second configuration (FIG. 13), as will be described below. Posterior and anterior components 126, 128 are movable by a linkage, which includes a first arm 132 and a second arm 134. First arm 132 includes a first end 138, a second end 140 and an outer surface 133. Second arm 134 includes a first end 142, a second end 144 and an outer surface 135. It is contemplated that components 126, 128 may be monolithically formed and/or be connected via a living hinge. It is further contemplated that anterior component 128 may be alternatively connected to posterior component 126 by integral connection, and/or fastening elements such as clips and/or screws.

First arm 132 comprises a first strut 148 and a second strut 150 disposed in parallel orientation. It is contemplated that struts 148, 150 can have alternate orientations, for example, converging, diverging and/or other angular orientations such as acute or obtuse, co-axial and/or may be offset or staggered and may extend in alternate configurations such as, for example, radius of curvature, offset and/or staggered. It is further envisioned that struts 148, 150 may have various cross section configurations, such as, for example, oval, oblong, triangular, rectangular, square, polygonal, irregular, uniform, non-uniform, variable, hollow and/or tapered.

Second arm 134 includes a first strut 152 and a second strut 154 disposed in parallel orientation. It is contemplated that struts 152, 154 can have alternate orientations for example, converging, diverging and/or other angular orientations such as acute or obtuse, co-axial and/or may be offset or staggered and may extend in alternate configurations such as, for example, radius of curvature, offset and/or staggered. It is further envisioned that struts 148, 150 may have various cross section configurations, such as, for example, oval, oblong, triangular, rectangular, square, polygonal, irregular, uniform, non-uniform, variable, hollow and/or tapered.

First arm 132 is rotatably connected to posterior component 126. The first end 138 of first arm 132 includes a pivot pin 166 for disposal with slot 168 and relative movement therein.

First arm 132 translates axially along slot 168 during expansion. Second arm 134 is rotatably connected to posterior component 126. The first end 142 of second arm 134 includes a pivot pin 167 for disposal with slot 168 and relative movement therein. Second arm 134 translates axially along slot 168 during expansion.

It is contemplated that arms 132, 134 may be disposed at alternate orientations, relative to longitudinal axis a-a, for example, perpendicular, converging, diverging and/or other angular orientations such as acute or obtuse, co-axial and/or may be offset or staggered. It is envisioned that arms 132, 134 may extend in alternate configurations such as, for example, radius of curvature, offset and/or staggered. It is further envisioned that arms 132, 134 may have various cross section configurations, such as, for example, oval, oblong, triangular, rectangular, square, polygonal, irregular, uniform, non-uniform, variable, hollow and/or tapered.

First and second arms 132, 134 are moveable by actuation of an actuator, such as, for example, a screw 194 disposed in the anterior component 128. Screw 194 includes a threaded outer surface 145. Screw 194 extends between first end 122 and second end 124 and is mounted in cavity 193 of anterior component 128. Screw 194 includes a first member, such as for example a wedge 170 and a second member, such as, for example a wedge 172.

Wedge 170 has a tapered outer surface and a threaded cylindrical inner surface 161. It is contemplated that wedge 170 can have alternate configurations, such as, oval, oblong, triangular, rectangular, square, polygonal, irregular, uniform, non-uniform, variable, hollow and/or tapered. It is contemplated that inner surface 161 can be smooth, even, arcuate, undulating and/or textured according to the requirements of a particular application. Wedge 170 is engageable with second shaft portion 194b of screw 194 and outer surface 133 of first arm 132.

Wedge 172 has a tapered outer surface and a threaded cylindrical inner surface 163. It is contemplated that wedge 172 can have alternate configurations, such as, oval, oblong, triangular, rectangular, square, polygonal, irregular, uniform, non-uniform, variable, hollow and/or tapered. It is contemplated that inner surface 163 can be smooth, even, arcuate, undulating and/or textured according to the requirements of a particular application. Wedge 172 is engageable with first shaft portion 194a of screw 194 and outer surface 135 of second arm 134.

Wedges 170, 172 are disposed at a mid portion and/or intermediate location of anterior component 128, and translate along screw 194 to facilitate expansion of components 126, 128. In one embodiment, wedge 170 has a first direction threading and wedge 172 has an opposite direction threading to move wedges 170, 172 along screw 194. In one embodiment, first shaft portion 194a has a first direction threading and second shaft portion 194b has an opposite direction threading to move wedges 170, 172 along screw 194. The threaded mating configuration of wedges 170, 172 and screw 194 is such that wedges 170, 172 are caused to diverge and space apart in translation from adjacent the mid portion and/or intermediate location of anterior component 128 towards ends 122, 124.

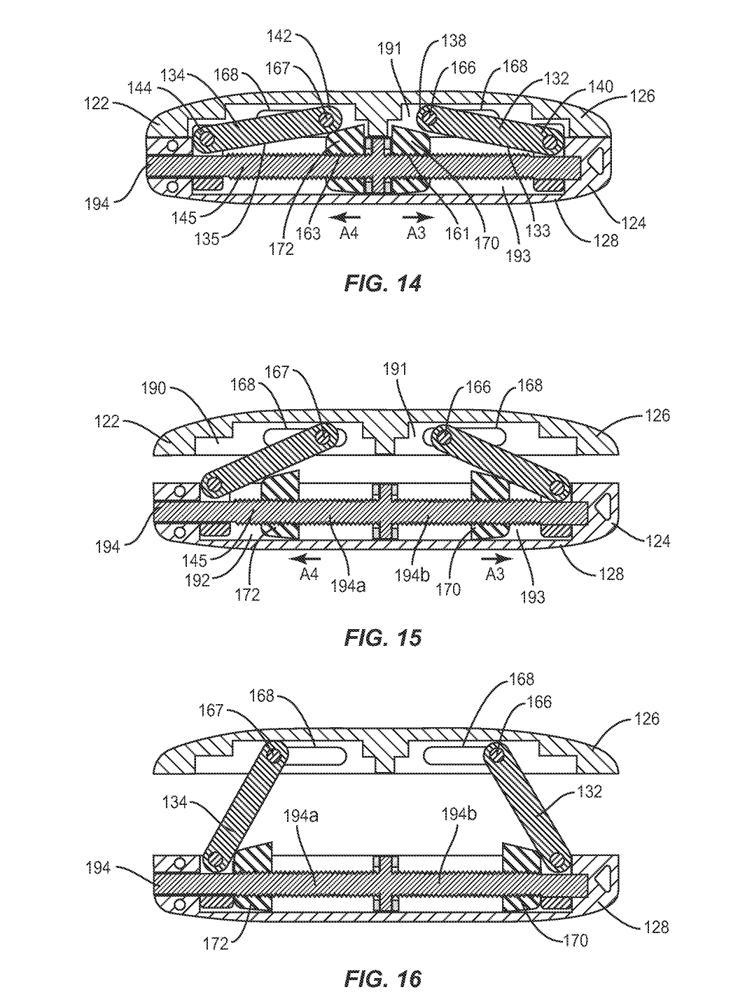

Wedge 170 is configured for axial movement in a first axial direction, as shown by arrow A3 in FIGS. 14 and 15, and wedge 172 is configured for axial movement in a second axial direction, as shown by arrow A4. Wedges 170, 172 translate along screw 194 in a diverging relation to engage outer surfaces 133, 135. As such, arms 132, 134 rotate relative to components 126, 128 and translate axially within slots 168, as described above, to move posterior component 126 and anterior component 128 between a first, collapsed configuration (FIG. 14) and a second, expanded configuration (FIG. 16).

In operation, as shown in FIGS. 14-21, intervertebral implant 20 including components 126, 128, is engaged for disposal between a first configuration and a second configuration such that intervertebral implant 20 expands in an intervertebral disc space. Intervertebral implant 20 is engaged with an instrument (not shown) to facilitate actuation of the component parts of intervertebral implant 20, according to the requirements of a particular surgical application.

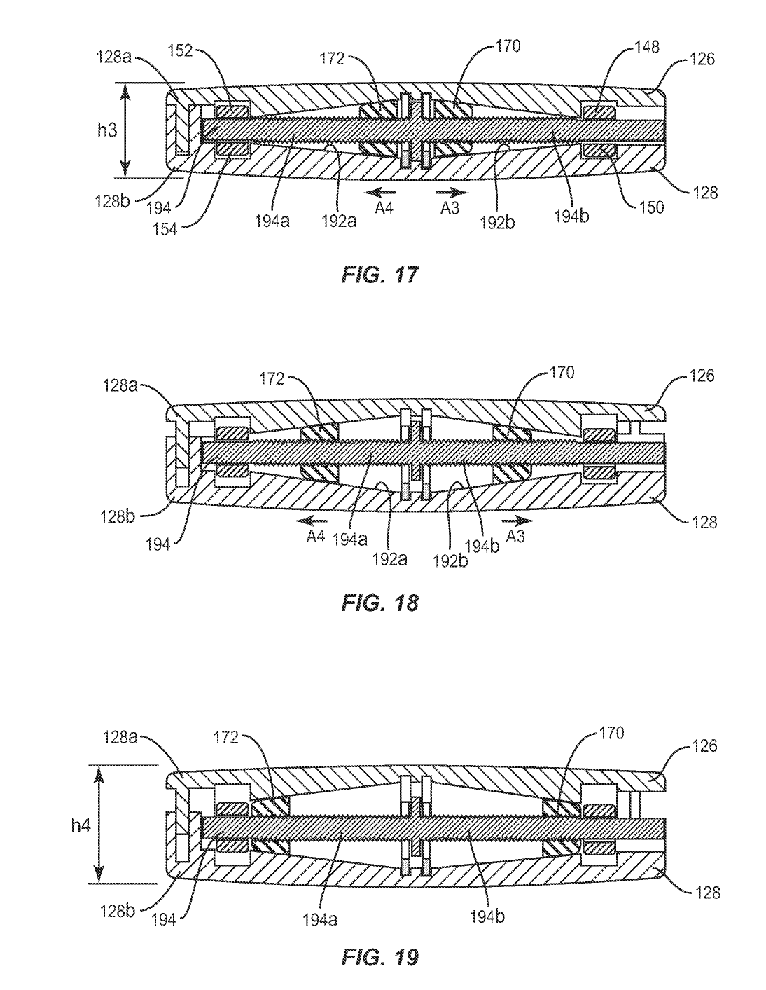

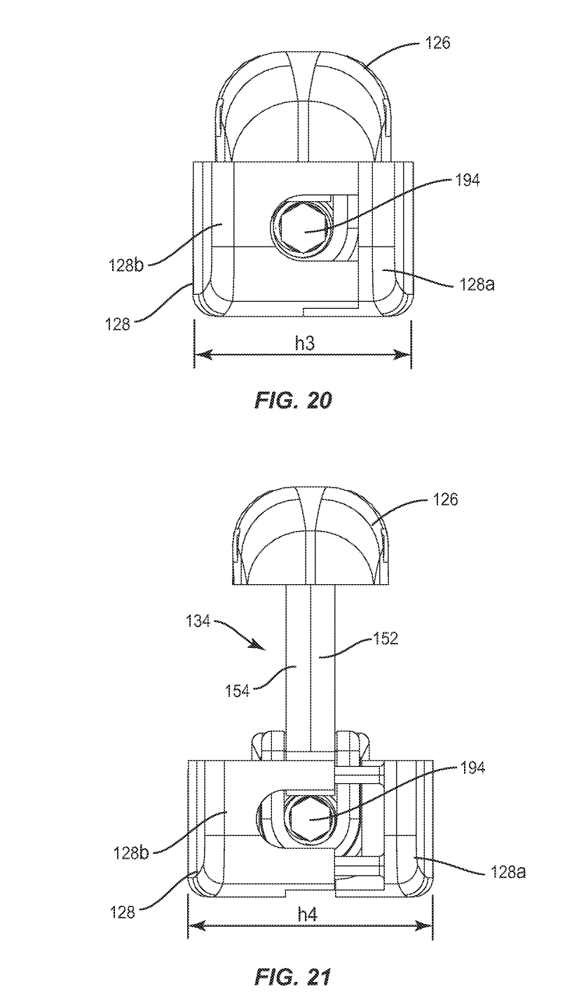

In a first configuration, such as for example, a collapsed configuration, as shown in FIGS. 14, 17 and 20, components 126, 128 are disposed in a low profile orientation such that wedges 170, 172 are disposed at a mid portion and/or intermediate location of anterior component 128. Posterior and anterior components 126, 128 are disposed in an engaging configuration such that the respective end surfaces of components 126, 128 are in flush contact. Members 128a, 128b are disposed in an engaging configuration such that the respective end surfaces of members 128a, 128b are in flush contact. Anterior component 128 has a height h3, as shown in FIGS. 17 and 20.

Upon desired positioning of intervertebral implant 20, according to the requirements of a particular surgical application, screw 194 is manipulated to move wedges 170, 172 axially. The instrument engages screw 194 for rotation in a clockwise direction. Screw 194 rotates such that outer surface 145 threadably engages inner surface 161 of wedge 170 and inner surface 163 of wedge 172. As screw 194 rotates, the threaded engagement with inner surfaces 161, 163 drive wedges 170, 172 in opposing axial directions, as shown by arrows A3, A4. Wedges 170, 172 translate along screw 194 in a diverging relation to engage outer surfaces 133, 135. As such, arms 132, 134 rotate relative to components 126, 128 and translate axially within slots 168, as described above, to move posterior component 126 and anterior component 128 between a first, collapsed configuration (FIG. 14) and a second, expanded configuration (FIG. 16). The linkage including arms 132, 134 cause component 126 to translate in a posterior direction relative to component 128 transverse to axis aa to space apart components 126, 128. Intervertebral implant 20 is expanded from an anterior to a posterior direction in an anterior-posterior expansion.

Simultaneously, as wedge 170 translates axially in the direction shown by arrow A3 and wedge 172 translates axially in the direction shown by arrow A4, wedges 170, 172 engage tapered portions 192a, 192b, respectively, to space apart members 128a, 128b to drive height expansion of anterior component 128 to a height h4, as shown in FIGS. 19 and 21. This configuration facilitates expansion of intervertebral implant 20 such that anterior component 128 has a greater rate and amount of expansion relative to posterior component 126 to induce lordosis. It is contemplated that in the expanded configuration, intervertebral implant 20 provides height restoration between vertebrae, decompression, restoration of sagittal balance and resistance of subsidence into endplates of vertebrae.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An intervertebral implant comprising:
a first component;
a second component;
a first arm extending between a first end and a second end;
a second arm extending between a first end and a second end, the first ends of the arms being engageable with the first component; and
an actuator disposed with the second component and including a first member and a second member, the members being configured for axial translation relative to the actuator,
wherein the first member translates in a first axial direction and is engageable with the first arm and the second member translates in a second axial direction and is engageable with the second arm such that the arms move the first component and the second component between a first, collapsed configuration and a second, expanded configuration.

2. An intervertebral implant as recited in claim 1 wherein at least one of the arms is rotatable relative to the first component.

3. An intervertebral implant as recited in claim 1 wherein the first end of the first arm includes a gear surface engageable with a gear surface of the first end of the second arm.

4. An intervertebral implant as recited in claim 1 wherein the first arm is rotatable in a counterclockwise direction relative to the first component and the second arm is rotatable in a clockwise direction relative to the first component to move the first component and the second component between the first, collapsed configuration and the second, expanded configuration.

5. An intervertebral implant as recited in claim 1 wherein at least one of the members includes a collar disposed about the actuator.

6. An intervertebral implant as recited in claim 1 wherein at least one of the arms define a bone graft receptacle.

7. An intervertebral implant as recited in claim 1 wherein the actuator includes a screw and at least one of the members defines a threaded inner surface configured for engagement therewith.

8. An intervertebral implant as recited in claim 1 wherein the first member and the second member are configured to converge during translation.

9. An intervertebral implant as recited in claim 1 wherein the first component has a first height and the second component has a second height, the second height being greater than the first height.

10. An intervertebral implant as recited in claim 1 wherein the first component comprises a posterior component having a first height and the second component comprises an anterior component having a second height, the second height being greater than the first height.

11. An intervertebral implant as recited in claim 1 wherein at least one of the members has a wedge configuration.

12. An intervertebral implant as recited in claim 1 wherein the first member has a tapered outer surface configured for slidable engagement with an outer surface of the first arm and the second member has a tapered outer surface configured for slidable engagement with an outer surface of the second arm.

13. An intervertebral implant as recited in claim 1 wherein at least one of the members has a tapered outer surface configured for slidable engagement with an inner surface of the second component.

14. An intervertebral implant as recited in claim 1 wherein the first component defines at least one slot configured for slidable movement of at least one of arms.

15. An intervertebral implant as recited in claim 1 wherein the first component defines a first slot and a second slot, the first end of the first arm being slidable within the first slot and the first end of the second arm being slidable within the second slot.

16. An intervertebral implant as recited in claim 15 wherein the first end of the first arm slides in a first axial direction along the first slot and the first end of the second arm slides in a second axial direction along the second slot.

17. An intervertebral implant as recited in claim 1 further defining a longitudinal axis, the components being expandable to the second configuration in at least one transverse direction relative to the longitudinal axis.

18. An intervertebral implant as recited in claim 1 wherein at least one of the members engage an inner surface of at least one of the first and second components to move the second component from a first collapsed height to a second expanded height.

19. An intervertebral implant comprising:
a posterior component extending between a first end and a second end, the posterior component including an outer surface and an inner surface, the posterior component defining a first slot and a second slot;
an anterior component extending between a first end and a second end, the anterior component including an outer surface and an inner surface;
a linkage including a first arm and a second arm, the first arm including a first end configured for axial movement within the first slot and a second end connected with the anterior component, the second arm including a first end configured for axial movement within the second slot and a second end connected with the anterior component;
an actuator including a longitudinal screw disposed with the anterior component, a first threaded collar disposed with the screw and a second threaded collar disposed with the screw,
wherein the screw is rotatable to translate the first collar in a first axial direction to engage the first arm such that the first arm rotates relative to the anterior component and the second collar in a second axial direction to engage the second arm such that the second arm rotates relative to the anterior component such that the components are movable in a first transverse orientation relative to the longitudinal axis between a first collapsed configuration and a second expanded configuration, and the anterior component is movable in a second transverse orientation between a first collapsed height and a second expanded height.

20. A method for treating a spine, the method comprising the steps of:
providing an intervertebral implant comprising:
a first component, a second component, a first arm extending between a first end and a second end, a second arm extending between a first end and a second end, the first ends of the arms being engageable with the first component, and an actuator disposed with the second component and including a first member and a second member, the members being configured for axial translation relative to the actuator;
introducing the intervertebral implant in a first, collapsed configuration along a substantially lateral approach of a body within an intervertebral space; and
engaging the actuator such that the first member translates in a first axial direction and is engageable with the first arm and the second member translates in a second axial direction and is engageable with the second arm such that the arms move the first component and the second component between the first, collapsed configuration and a second, expanded configuration.
